(19) United States
(12) Patent Application Publication
(10) Pub. No.: US 2012/0203291 A1
(43) Pub. Date: Aug. 9, 2012

(54) SPINAL FIXATION ROD ADJUSTMENT SYSTEM AND METHOD

(75) Inventor: Martin Boulaine, Carlsbad, CA (US)

(73) Assignee: Alphatec Spine, Inc., Carlsbad, CA (US)

(21) Appl. No.: 13/363,877

(22) Filed: Feb. 1, 2012

Related U.S. Application Data
(60) Provisional application No. 61/439,051, filed on Feb. 3, 2011.

(51) Int. Cl. A61B 17/56

(52) U.S. Cl. 606/86 R; 606/86 A

(57) ABSTRACT

An instrument for adjusting a fixation rod within a head member of a screw assembly includes a distal portion, a first arm, a first recess, and an actuation member. The distal portion includes a rod engagement surface that engages the fixation rod. The first arm extends from the distal portion towards the head member at a first angle. The first recess is disposed along the first arm at a first distance from the rod engagement surface. The actuation member extends proximally from the distal portion and positions the first recess relative to a first projection on the head member.

### CROSS-REFERENCE TO RELATED APPLICATIONS

This applications claims priority to U.S. Provisional Application Ser. No. 61/439,051 filed on Feb. 3, 2011 which is incorporated herein by reference in its entirety.

### FIELD

The present disclosure generally relates to the field of spinal orthopedics, and more particularly to systems and methods for adjusting a spinal fixation rod in a fixation device.

### BACKGROUND

The spine is a flexible column formed of a plurality of bones called vertebrae. The vertebrae are hollow and piled one upon the other, forming a strong hollow column for support of the cranium and trunk. The hollow core of the spine houses and protects the nerves of the spinal cord. The different vertebrae are connected to one another by means of articular processes and intervertebral, fibrocartilaginous bodies. Various spinal disorders may cause the spine to become misaligned, curved, and/or twisted. It is often necessary to surgically correct and stabilize spinal curvatures or to facilitate spinal fusion between two or more adjacent vertebrae.

One procedure for treating spinal disorders involves attaching a rigid system of screws and rods to the posterior side of the vertebrae. A patient may be positioned to permit surgical access to the spinal area. Once the spine is exposed, a series of screw assemblies may be inserted into each of the vertebrae to permit attachment of fixation rods to the vertebrae. The screw assemblies may include poly-axial screws that are fixed to the pedicles of the vertebrae. The screw assemblies may also include screw heads with a threaded channel for receiving the rods. The fixation rods may be shaped to a predetermined alignment and curvature depending on the anatomy of the patient. The fixation rods may be loosely fitted into the screw heads prior to fixation. Once the fixation rods are inserted into the screw heads, the fixation rods must be fully seated in the screw heads before the locking setscrews can be installed to lock the fixation rods within the screw heads.

Generally speaking, the fixation rods and screw assemblies are made from high-strength materials that resist deformation and may require high levels of force to properly seat the rods in the heads. One instrument commonly used to fully seat the spinal fixation rod is a fixation rod adjustment instrument commonly referred to as a “rod rocker.” FIG. 1 illustrates a prior art rod rocker 100. The rod rocker 100 may include two shanks 102 and 104 pivotably coupled with a pin or screw 106 similar to a scissors or pliers. The shanks 102 and 104 may include handle portions 108 and 110 on a proximal end and a pair of extended portions 112 and 114 on a distal end. When closed, the extended portions 112 and 114 form a cavity 116 configured to receive a head portion 204 of a screw assembly 200 as depicted in FIGS. 2A-2C. A pair of pins 118 project inwards towards the center of the cavity 116. The pins 118 may be configured to engage with corresponding receptacles 208 on the head portion 204 as depicted in FIGS. 2A-2C. A heel portion 120 of the cavity 116 may be formed when the extended portions 112 and 114 are clamped together in a closed configuration. The cavity may be enlarged by moving the extended portions 112 and 114 to an open configuration. The heel portion 120 may be configured to engage with a fixation rod 300 as depicted in FIGS. 2A-2C.

Continuing with FIGS. 2A-2C, actuation of the rod rocker 100 with respect to an implanted screw assembly 200 and fixation rod 300 is shown in greater detail. The screw assembly 200, including a threaded portion 202 and the head portion 204, is attached to a vertebra 400. The screw assembly 200 may be, for example, a poly-axial screw assembly. The head portion 204 may be configured to rotate and swivel about a rounded portion (not shown) on the proximal end of the threaded portion 202 to facilitate attachment of the fixation rod 300. A channel 206 running through the interior of the head portion 204 is configured to receive the fixation rod 300. The receptacles 208 on an outer surface of the head portion 204 are configured to receive the pins 118 of the rod rocker 100. In FIG. 2A, the fixation rod 300 has been loosely fixed within the head portion 204 of the screw assembly 200. The rod rocker 100 is positioned adjacent the screw assembly 200 in the open configuration to permit the extended portions 112 and 114 to extend around the head portion 204.

Referring now to FIG. 2B, the extended portions 112 and 114 close around the head portion 204 of the screw assembly 200. The pins 118 are engaged with the receptacles 208. Thus, the rod rocker 100 may pivot about the pins 118 as the cavity 116 surrounds the head portion 204. When the rod rocker 100 is rotated, the heel portion 120 begins to contact the fixation rod 300. The heel portion 120 may be used to apply a force to the fixation rod 300 to position the fixation rod 300 deeper into the channel 206. For example, the operator may apply a force to the handle portions 108 and 110 such that the heel portion 120 transfers some amount of the force to the fixation rod 300 while the pins 118 continue to grasp the receptacles 208 on the head portion 204. The extended portions 112 and 114 provide leverage to increase the amount of the force applied to the fixation rod 300.

In FIG. 2C, the rod rocker 100 has fully seated the fixation rod 300 within the head portion 204. Force may continue to be applied to the rod rocker 100 to maintain the fixation rod 300 within the head portion 204 while a locking screw 210 is threaded onto corresponding threads 212 within the channel 206. Once the locking screw 210 has been fully tightened, the rod rocker 100 may be opened to release the pins 118 from the receptacles 208.

Typically, a rod rocker, such as the rod rocker 100, includes a number of different parts. For example, many rod rockers include multiple shanks, handles, and swivel pins that enable the rod rocker to open around the head portion of a screw assembly so that the pins can engage the receptacles on the head portion. The pins are formed or machined from the rod rocker or alternatively attached by welding or friction fitting. These many parts increase the complexity and cost of producing the rod rocker. In addition, some of the parts, such as the pins, may inhibit maneuvering the rod rocker within a surgical area. For example, the pins may become entangled with tissue in the surgical area. The pins may also fracture or break due to high stresses associated with maneuvering the fixation rod. Furthermore, the amount of force applied by the rod rocker may be limited because the pins provide a single pivot axis about which the rod rocker may rotate. For example, the amount of leverage provided by the extended portions is also limited because the distance between the heel portion and the pivot point is fixed.

### SUMMARY

An instrument for adjusting a fixation rod within a head member of a screw assembly includes a distal portion, a first arm, a first recess, and an actuation member. The distal portion includes a rod engagement surface that engages the fixation rod. The first arm extends from the distal portion towards the head member at a first angle. The first recess is disposed along the first arm at a first distance from the rod engagement surface. The actuation member extends proximally from the distal portion and positions the first recess relative to a first projection on the head member.

In other features, a second arm extends parallel to the first arm to form a pair of arms. A second recess is disposed along the second arm at the first distance from the rod engagement surface. The actuation member positions the second recess relative to a second projection on the head member. In still other features, the first and second arms form a U-shaped cavity that receives the head member. The first and second recesses mate with the first and second projections extending from the head member.

In yet other features, the first arm is perpendicular to the distal portion. The actuation member pivots the distal portion about the first projection to seat the fixation rod within the head member. The distal portion and the first arm form an L-shaped profile. The rod engagement surface includes a concave profile configured to engage a convex profile of the fixation rod. A plurality of recesses is disposed along the first arm at a plurality of distances from the rod engagement surface. Each of the plurality of recesses provides a plurality of magnitudes of leverage to position the fixation rod within the head member.

A system for adjusting a fixation rod of a spinal fixation system includes a screw assembly and an instrument. The screw assembly includes a threaded shaft with a distal end that attaches to a vertebra and a proximal end and a head member including a channel for receiving the fixation rod and an outer surface including a pair of projections. The instrument includes a distal portion, a pair of arms with a plurality of recesses, and an actuation member. The distal portion includes a concave distal surface for engagement with a convex surface of the fixation rod. The pair of arms extends perpendicularly from the distal portion towards the head member. The plurality of recesses is disposed along proximal surfaces of the pair of arms and include a profile that mates with the pair of projections on the head member. The actuation member extends proximally from the distal portion and applies a force to pivot the distal portion about the projections and seat the fixation rod within the head member.

In other features, inner surfaces of the pair of arms are separated by a width W1. The pair of projections extends from recessed portions of the outer surface separated by a width W2 that is less than width W1. Outer surfaces of the projections are separated by a width W3 that is greater than the width W1.

A method of adjusting a fixation rod relative to a head member of a screw assembly with an adjustment instrument includes the steps of engaging a distal portion the rocker head with the fixation rod; sliding a pair of arms extending perpendicularly from the distal portion relative to the head member; engaging recesses on proximal surfaces of the pair of arms with a mating pair of projections on the head member; and applying a force on an actuation member extending proximally from the distal portion to position the fixation rod within the head member.

In other features, the step of engaging the distal portion includes engaging a concave distal surface with a convex surface of the fixation rod. The method includes the step of selecting from a plurality of pairs of recesses at a plurality of distances from the distal portion to change the leverage of the adjustment instrument.

### BRIEF DESCRIPTION OF THE DRAWINGS

FIG. 1 is an elevational side view of a prior art spinal rod adjustment instrument.

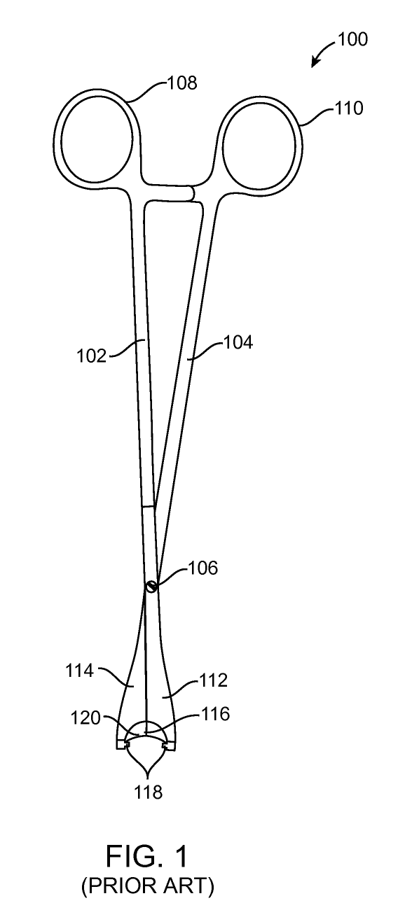

FIGS. 2A-2C are perspective views of a prior art spinal rod adjustment system and method.

FIG. 3 is a perspective view of a spinal rod adjustment instrument according to the principles of the present disclosure.

FIG. 4 is an elevational side view of the spinal rod adjustment instrument according to the principles of the present disclosure.

FIG. 5 is an elevational front view of the spinal rod adjustment instrument according to the principles of the present disclosure.

FIG. 6 is a perspective view of a head portion for a screw assembly according to the principles of the present disclosure.

FIG. 7 is a partial cross-sectional view of the screw assembly and a fixation rod according to the principles of the present disclosure.

FIGS. 8A-8D are perspective views of a spinal fixation rod adjustment system and method according to the principles of the prior art.

### DETAILED DESCRIPTION

The spinal fixation rod adjustment system and methods described herein simplify the manufacturing process and reduce costs associated with the various parts of prior rod rockers. In addition, the system and methods reduce the risk of entanglement with tissue while maneuvering the rod rocker within the surgical area. Furthermore, the system and methods provide a more versatile instrument than prior rod rocker and screw head systems and methods by providing a choice of multiple pivot points.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

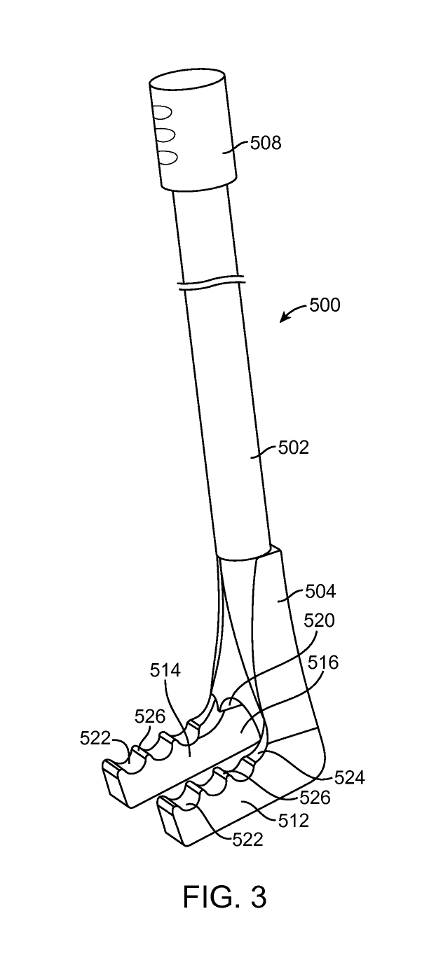

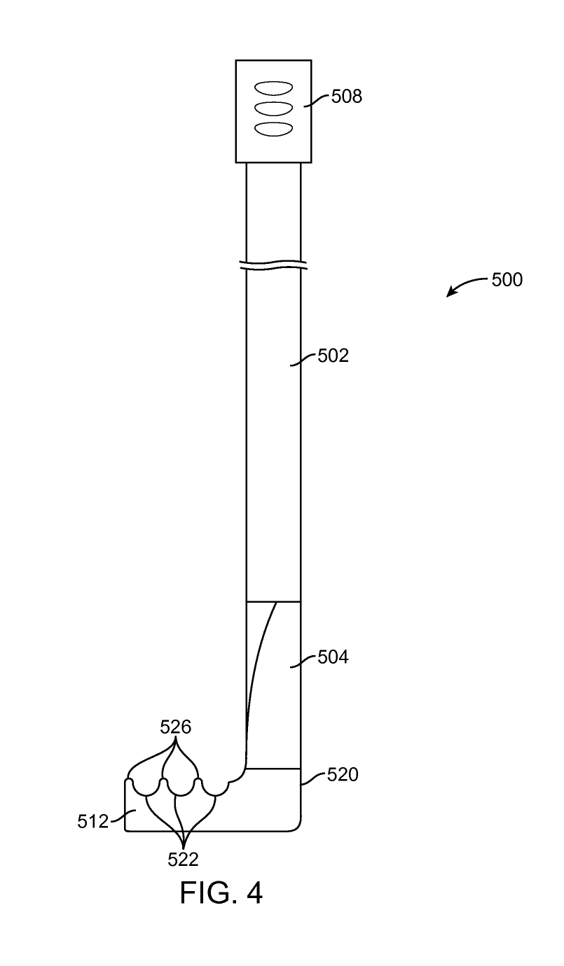

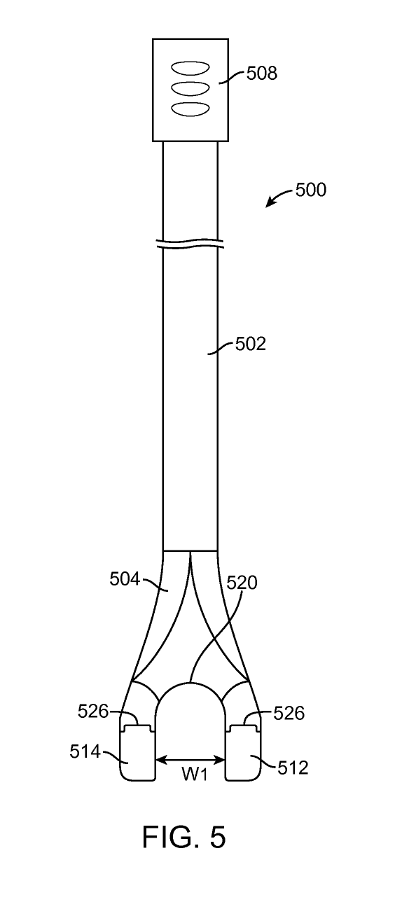

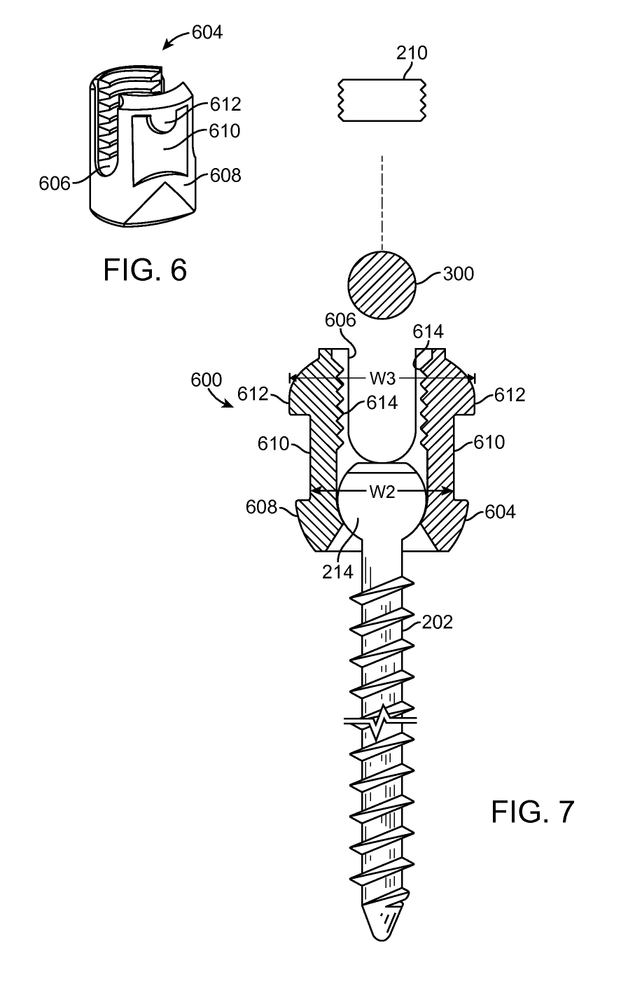

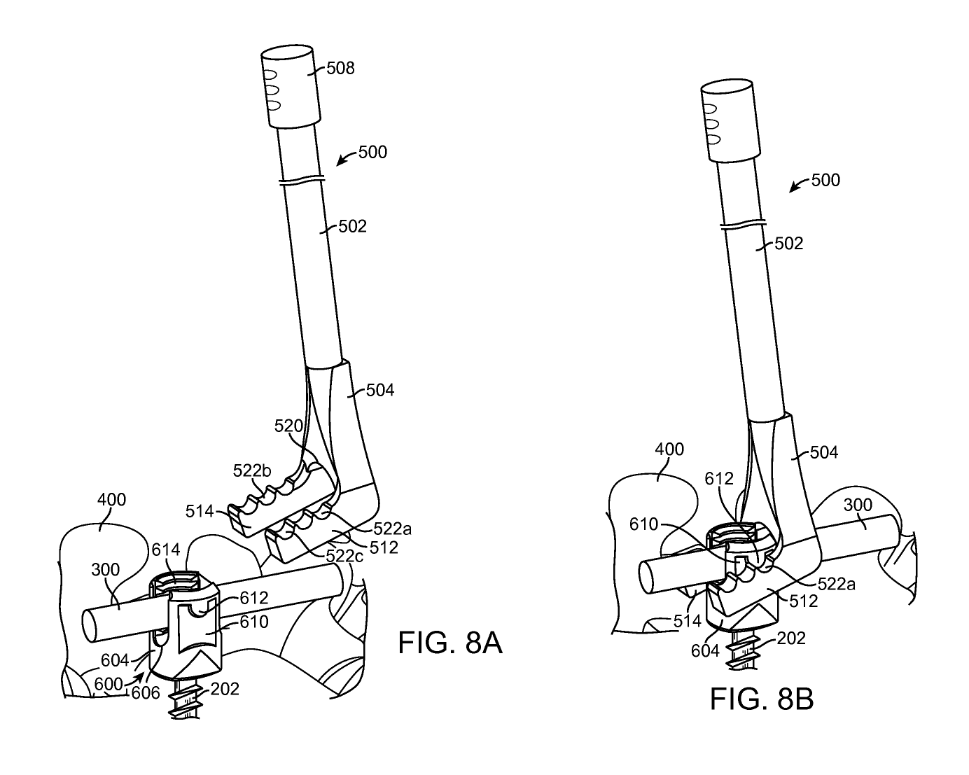

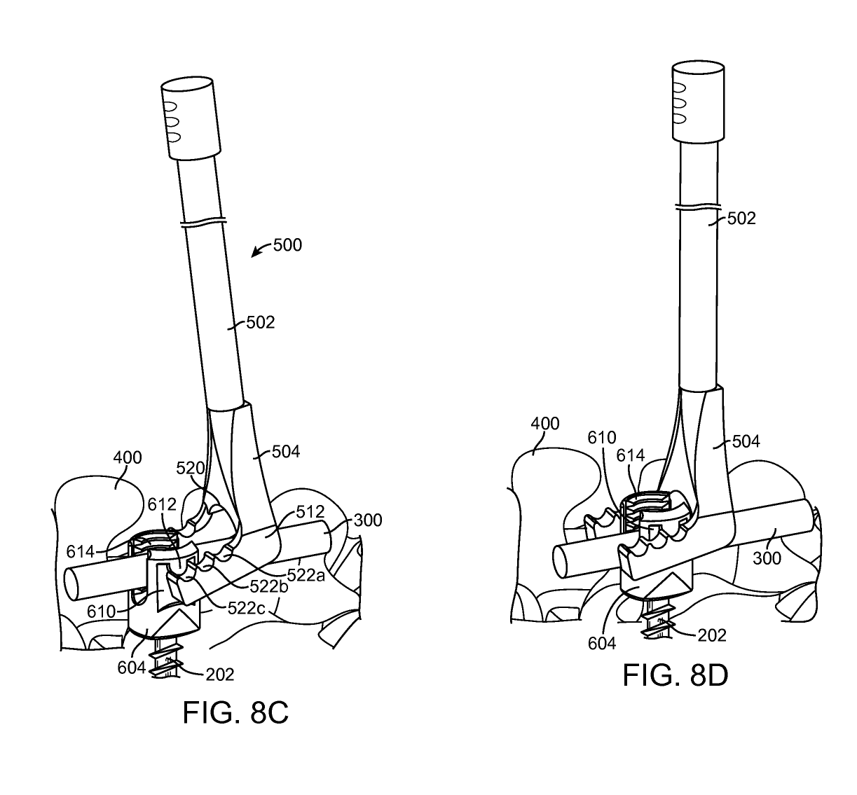

Referring now to FIGS. 3-5, an instrument 500 for adjusting a fixation rod within a head member of a screw assembly according to the principles of the present disclosure is shown. The instrument 500 may include a shank 502 with a distal portion 504 attached to a distal end and an actuation member 508 attached to a proximal end. The actuation member 508 may be used to apply a force to seat a spinal fixation rod 300 as depicted in FIGS. 8A-8D. A pair of arms 512 and 514 extends away from the distal portion 504. The arms 512 and 514 may form an L-shaped profile with the distal portion 504. For example, the arms 512 and 514 may extend substantially perpendicular from the distal portion 504. The arms 512 and 514 may extend at various angles from the distal portion 504. The arms 512 and 514 form a U-shaped cavity 516 configured to engage the fixation rod 300 and to receive a head portion 604 of a screw assembly 600 as shown in FIGS. 6-7.

The interior surfaces of the arms 512 and 514 may be separated by a width W1 that enables the distal portion 504 to slidably engage with the head portion 604. Although two arms 512 and 514 are depicted, one arm may be sufficient to perform the adjustments described herein. A rod engagement surface or heel portion 520 of the cavity 516 may be configured to engage with a convex surface of the fixation rod 300 as depicted in FIGS. 8A-8D. For example, the rod engagement surface may include a concave surface. One or more pairs of recesses 522 may be disposed along a proximal surface 524 of the arms 512 and 514. The recesses 522 may be separated by ridges 526 and configured to engage with corresponding portions of the head portion 604 of the screw assembly 600 as depicted in FIGS. 8A-8D.

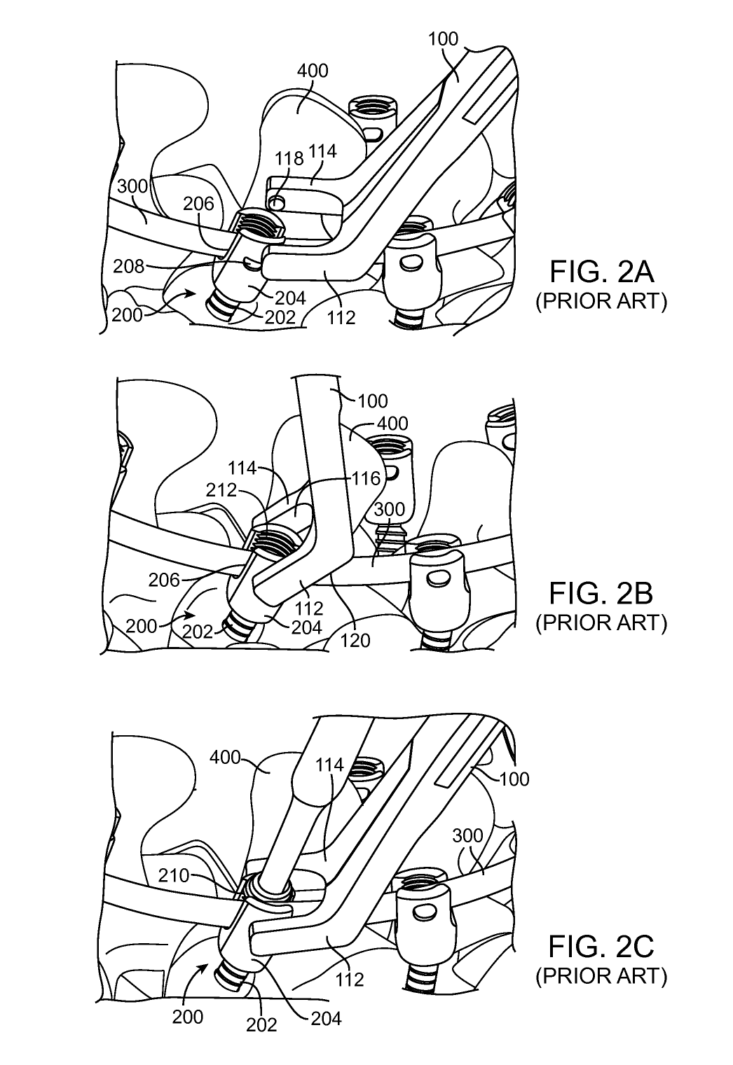

Referring now to FIGS. 6 and 7, the head portion 604 may be configured to attach to the threaded portion 202 of the screw assembly 200 in FIGS. 2A-2C or any other screw according to the prior art. For example, the head portion 604 may couple with a rounded portion 214 on the proximal end of the threaded portion 202 in a poly-axial screw configuration. A channel 606 running through the interior of the head portion 604 is configured to receive the fixation rod 300. An outer surface 608 of the head portion 604 includes two recessed surfaces 610 that are separated by the width W2 corresponding to the width between the arms 512 and 514 of the distal portion 504. The width W2 may be less than the width W1 to permit the arms 512 and 514 to slide around the head portion 604. A projection 612 protrudes from each of the recessed surfaces 610. For example, the projection 612 may be a half-cylinder shaped projection configured to engage with the recesses 522 on the arms 512 and 514 of the instrument 500 as shown in FIGS. 8A-8D. The outer surfaces of the projections 612 may be separated by a width W3 that is greater than the width W1. Once the fixation rod 300 is seated in the channel 606, the setscrew 210 may be attached to compress the fixation rod 300 and rounded portion 214 of the threaded portion 202 together with the head portion 604. Alternatively, a bushing (not shown) may be disposed between the fixation rod 300 and the rounded portion 214 as known in the art.

Referring now to FIGS. 8A-8D, a system and method for adjusting the fixation rod 300 relative to the screw assembly 600 is shown. The screw assembly 600, including the threaded portion 202 and the head portion 604, has been attached to the vertebra 400. For example, the screw assembly 600 may be a poly-axial screw assembly. The head portion 604 is configured to rotate and swivel about the rounded portion (not shown) on the proximal end of the threaded portion 202 to facilitate attachment of the fixation rod 300. In FIG. 8A, the fixation rod 300 has been loosely fixed within the head portion 604 of the screw assembly 600. For example, only two threads 614 may be visible above the fixation rod 300 before the fixation rod 300 is seated within the head portion 604. The instrument 500 is positioned adjacent the screw assembly 600.

Referring now to FIG. 8B, the arms 512 and 514 of the instrument 500 slide relative to the recessed surfaces 610 to encompass the head portion 604 of the screw assembly 600. The recesses 522 engage with the projections 612 on the head portion 604 to provide a pivot about which the instrument 500 may rotate. For example, in FIG. 8B, the projections 612 engage with a first pair of recesses 522a closest to the heel portion 520 of the distal portion 504. For example, the first pair of recesses 522a may be a first distance from the heel portion 520. Alternatively, the projections 612 may engage with any of the first, second, and third pairs of recesses 522a-c. The second pair of recesses 522b may be a second distance from the heel portion that is greater than the first distance. The third pair of recesses 522c may be a third distance from the heel portion that is greater than the second distance. For example in FIG. 8C, the projections 612 engage with the third pair of recesses 522c furthest from the heel portion 520 of the distal portion 504. Thus, the instrument 500 may be easily adjusted to control the amount of leverage provided by the arms 512 and 514 by choosing the appropriate pair of recesses 522.

Although three pairs of recesses 522 are depicted in the figures, additional or fewer recesses 522 may be appreciated by those skilled in the art. Thus, the instrument 500 may pivot about the projections 612 as the cavity 516 surrounds the head portion 604. The heel portion 520 contacts the fixation rod 300 as the instrument 500 is rotated. The heel portion 520 may be used to apply a force to the fixation rod 300 to position the rod 300 deeper into the channel 606. For example, the operator may apply a force to the actuation member 508 such that the heel portion 520 transfers some amount of the force to the fixation rod 300 while the recesses 522 continue to engage the projections 612 on the head portion 504.

Similarly, although a pair of arms 512 and 514 is depicted in the figures, a single arm may perform the same function. However, using the pair of arms 512 and 514 provides greater strength and stability to the construct. For example, the first arm 512 may pivot about one of the projections 612 as the distal portion surrounds the head portion 604. The heel portion 520 contacts the fixation rod 300 as the instrument 500 is rotated. The heel portion 520 may be used to apply a force to the fixation rod 300 to position the rod 300 deeper into the channel 606. For example, the operator may apply a force to the actuation member 508 such that the heel portion 520 transfers some amount of the force to the fixation rod 300 while the recesses 522 continue to engage the projections 612 on the head portion 504.

In FIG. 8D, the instrument 500 has fully seated the fixation rod 300 within the head portion 604. For example, four threads 614 may be visible above the fixation rod 300 after the fixation rod 300 is seated within the head portion 604. Force may continue to be applied to the instrument 500 to maintain the fixation rod 300 within the head portion 604 while the locking screw 210 of FIGS. 2A-2C may be threaded onto corresponding threads 614 within the channel 606. Once the locking screw has been fully tightened, the instrument 500 may slide away from the recessed surfaces 610 to be removed from the head portion 604.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

The invention claimed is:

1. An instrument for adjusting a fixation rod within a head member of a screw assembly, comprising:
a distal portion including a rod engagement surface that engages the fixation rod;
a first arm extending from the distal portion towards the head member at a first angle;
a first recess on the first arm at a first distance from the rod engagement surface; and
an actuation member extending proximally from the distal portion that positions the first recess relative to a first projection on the head member.

2. The instrument of claim 1, further comprising:
a second arm extending parallel to the first arm to form a pair of arms; and
a second recess on the second arm at the first distance from the rod engagement surface,
wherein the actuation member positions the second recess relative to a second projection on the head member.

3. The instrument of claim 2, wherein the first and second arms form a U-shaped cavity that receives the head member.

4. The instrument of claim 2, wherein the first and second recesses mate with the first and second projections extending from the head member.

5. The apparatus of claim 1, wherein the first arm is perpendicular to the distal portion.

6. The instrument of claim 1, wherein the actuation member pivots the distal portion about the first projection to seat the fixation rod within the head member.

7. The instrument of claim 1, wherein the distal portion and the first arm form an L-shaped profile.

8. The instrument of claim 1, wherein the rod engagement surface includes a concave profile configured to engage a convex profile of the fixation rod.

9. The instrument of claim 1, further comprising a plurality of recesses on the first arm at a plurality of distances from the rod engagement surface.

10. The instrument of claim 10, wherein each of the plurality of recesses provides a plurality of magnitudes of leverage to position the fixation rod within the head member.

11. A system for adjusting a fixation rod of a spinal fixation system, comprising:
a screw assembly having
a threaded shaft with a distal end that attaches to a vertebra and a proximal end; and
a head member including a channel for receiving the fixation rod and an outer surface including a pair of projections; and
an instrument having
a distal portion including a concave distal surface for engagement with a convex surface of the fixation rod;
a pair of arms extending perpendicularly from the distal portion towards the head member;
a plurality of recesses on proximal surfaces of the pair of arms, said plurality of recesses including a profile that mates with the pair of projections on the head member; and
an actuation member extending proximally from the distal portion that applies a force to pivot the distal portion about the projections and seat the fixation rod within the head member.

12. The system of claim 11, wherein inner surfaces of the pair of arms are separated by a width W1.

13. The system of claim 12, wherein the pair of projections extends from recessed portions of the outer surface separated by a width W2 that is less than width W1.

14. The system of claim 13, wherein outer surfaces of the projections are separated by a width W3 that is greater than the width W1.

15. A method of adjusting a fixation rod relative to a head member of a screw assembly with an adjustment instrument, comprising:
engaging a distal portion the rocker head with the fixation rod;
sliding a pair of arms extending perpendicularly from the distal portion relative to the head member;
engaging recesses on proximal surfaces of the pair of arms with a mating pair of projections on the head member;
applying a force on an actuation member extending proximally from the distal portion to position the fixation rod within the head member.

16. The method of claim 15, wherein engaging the distal portion includes engaging a concave distal surface with a convex surface of the fixation rod.

17. The method of claim 15, further comprising selecting from a plurality of pairs of recesses at a plurality of distances from the distal portion to change the leverage of the adjustment instrument.
